(19) United States
(12) Patent Application Publication
(10) Pub. No.: US 2006/0030877 A1
(43) Pub. Date: Feb. 9, 2006

(54) Distal filtration devices and methods of use during aortic procedures

(76) Inventors: Lorraine M. Martinez, Fremont, CA (US); Brian Nuel, Mountain View, CA (US); Peter Thornton, Los Altos, CA (US); Gary Sunseri, Hollister, CA (US); Jobert Balceta, San Jose, CA (US)

Correspondence Address: LYON & LYON LLP, 633 WEST FIFTH STREET, SUITE 4700, LOS ANGELES, CA, 90071, US

(21) Appl. No.: 11/241,072

(22) Filed: Sep. 30, 2005

Related U.S. Application Data
(62) Division of application No. 10/066,504, filed on Jan. 30, 2002, now abandoned.

(51) Int. Cl. A61M 29/00

(52) U.S. Cl. 606/200

(57) ABSTRACT

Methods and devices for surgical repair of an aortic aneurysm are described. The devices comprise an elongate member having one or more expandable filters or membranes mounted at a distal end. In certain embodiments, the devices also include aspiration and flushing capability to assist in removal of embolic debris. In use, the filter is inserted and expanded downstream of an aortic aneurysm. The aneurysm is repaired and the filter captures emboli dislodged during the repair. The filter is then collapsed and removed from the aorta.

### FIELD OF THE INVENTION

The present invention relates generally to medical devices useful for capturing embolic material in blood vessels. More specifically, the devices and methods provide a vessel filtering system for temporary deployment in arteries and veins, such as the aorta, the iliac arteries, and the femoral arteries. The devices also include aspiration and flushing capability to assist in removal of embolic material generated during vascular procedures.

### BACKGROUND OF THE INVENTION

Atherosclerosis is the underlying cause of a majority of disorders involving the aorta, such as aneurysm, dissection, and rupture. The abdominal aorta is most commonly involved and often requires surgical treatment, such as atherectomy, aorto-femoral bypass, repair of abdominal aortic aneurysm (AAA), and repair of aortic dissection. In abdominal aortic aneurysm, for example, more than 95 percent of the cases are due to atherosclerosis. Manipulation of the diseased aorta during surgeries often generates embolic debris, such as calcium, atheromatous plaque, thrombi, and vascular tissue. These emboli travel downstream to occlude smaller vessels that supply, for example, the legs, kidneys, or intestines, causing ischemia or infarction. The incidence of atheroembolism to the lower extremities due to aortic surgeries is reported to be in the range of 2-29%, with over 30% of those patients requiring amputation, and post-operative mortality rates of those patients of approximately 25% in 30-day.

During a typical abdominal aortic aneurysm repair, for example, the abdominal aorta is first exposed and mobilized through a midline abdominal incision as described in Sabiston, Textbook of Surgery, 12^{th} edition, 1981. Arterial clamps are placed on the aorta above the region of interest and on the iliac arteries below. The inferior mesentery artery, which is usually obliterated at its origin from the aorta, is ligated and divided. The aneurysm is then incised. The anterior portion of the aneurysm and thrombus, if present, are removed. Excess aneurismal tissue is trimmed away. A preclotted prosthetic graft of woven Dacron is inserted and sutured end to end into the aorta. The remaining aneurysmal wall is then sutured around the Dacron graft, the posterior peritoneum is closed, and arterial clamps are released to re-establish blood flow.

During the procedure, generation of embolic debris typically occurs during incision, clamping and unclamping of the aorta. Currently there are a few methods used by the surgeons to decrease embolic load to the distal arteries. One method involves controlling the embolic load by cross-clamping an artery distal to the arteriotomy or lesion during the procedure. This clamping procedure eliminates blood flow and prevents emboli from flowing into the lower extremities during the operation. However, clamping itself also generates emboli if the clamp is placed onto a diseased artery. Embolic load to the extremities can be assessed by performing ultrasound or doppler pre and postoperatively to monitor pedal and digital perfusion.

Therefore, devices and methods are needed to protect against distal embolization during vascular procedures, especially involving the aorta, thereby minimizing end organ ischemia and infarction.

### SUMMARY OF THE INVENTION

The present invention provides vascular filtration devices and methods useful for placement downstream of a vascular lesion or arteriotomy where embolic debris, such as calcium, thrombi, atherosclerotic plaque, and tissue fragments, generated during the vascular procedure is captured before traveling downstream into other organs, e.g., the legs or kidneys.

In a first embodiment, the filtration device includes a collapsible filter mounted on a distal end of an elongate member, e.g., a wire, adapted for insertion into a vessel, such as an aorta. The filter is collapsed by advancing a sheath over the filter and is expanded by removing the sheath proximally.

In another embodiment, the device includes a distal capture sheath. The filter and the sheath are fixed proximally to a handle. The capture sheath is attached proximally to a wire and is movable relative to the handle. The filter is collapsed into the sheath by pulling the wire and the capture sheath proximally. The filter is expanded by advancing the wire and capture sheath distally.

In another embodiment, the device includes aspiration capability. The filter can take on a windsock design with an open tip at its distal end that allows aspiration into the sheath. This design is particularly helpful in procedures where a large embolic load is generated. Aspiration of the embolic debris as it is filtered prevents clogging of the filter and leakage of embolic debris from the filter.

In other embodiments, the device includes a rotation mechanism that allows closure of the filter. One mechanism includes a helical strut where one end of the filter is fixed to a first elongate member, such as a wire, and the other end of the filter is fixed to a second elongate member. When one wire is held stationary, the other wire rotates clockwise or counterclockwise to close the strut and the filter.

In another embodiment, the device includes a second collapsible filter mounted distal to the first collapsible filter. Each filter is independently collapsed and expanded by separate mechanisms. For example, advancing a sheath distally collapses the first filter, and pulling a wire proximally collapses the second filter. This design is particularly useful in vascular procedures where blood flow occurs in both directions, i.e., antegrade and retrograde. The filter can be independently closed or opened depending on the direction of blood flow.

In using the filtration devices to prevent distal embolization during vascular procedures, for example abdominal aortic aneurysm repair, the distal end of the elongate tubular member carrying the collapsed filter is inserted through an incision in a peripheral artery, e.g., the femoral artery, and advanced in a retrograde direction to position in the abdominal aorta above or below the renal arteries, the iliac arteries, or the femoral arteries downstream of the arteriotomy. The filter is expanded. The abdominal aorta is then exposed and mobilized through a midline abdominal incision. Arterial clamps are placed on the aorta above the region of interest and on the iliac arteries below. The inferior mesentery artery, which is usually obliterated at its origin from the aorta, is ligated and divided. The aneurysm is then incised. The anterior portion of the aneurysm and thrombus, if present, are removed. Excess aneurismal tissue is trimmed away. A preclotted prosthetic graft of woven Dacron is inserted and sutured end to end into the aorta. The remaining aneurysmal wall is then sutured around the Dacron graft, the posterior peritoneum is closed, and arterial clamps are released to re-establish blood flow. Debris generated during the procedure, especially during release of the clamps, is captured by the filter, thereby preventing distal embolization to the lower extremities and/or the kidneys. The filter that has captured the embolic debris is collapsed and removed.

In another method, the filtration device is introduced laporscopically into the abdominal aorta through a port access or minimally invasive incision. An abdominal port of approximately 20 mm is used to gain access to the aorta. An incision is made and a purse string is placed on the aorta. An introducer port is inserted and the filter is introduced into the aorta in an antegrade or retrograde direction distal to the aorteriotomy. After the vascular procedure, the filter is collapsed and removed through the introducer port.

In another method, during vascular procedures where an arterial shunt is required to maintain peripheral circulation, the filtration device is inserted directly into the shunt through a branching side port and is deployed during surgery to capture embolic debris. Alternatively, during aortic aneurysm repair, for example, the filter is inserted through the Dacron graft and is deployed in the aorta or the iliac arteries to capture embolic debris. After the arterial clamp is released and the filter captures embolic debris, the filter is collapsed and removed. The insertion site on the Dacron graft is then repaired.

It will be understood that there are several advantages to using the filtration devices and methods described herein. For example, the devices and methods (1) are particularly suited for temporary filtration of blood in any vessel, especially the aorta, to entrap embolic debris, thereby minimizing organ damage associated with distal embolization, (2) can withstand high arterial blood flow for an extended time, (3) includes a mesh that is porous enough to allow adequate blood flow in a blood vessel while capturing emboli, (4) provide aspiration capabilities to remove embolic debris especially during a large embolic load, (5) are able to capture emboli when blood flow occurs in retrograde and antegrade directions, (6) can be inserted directly into an arterial shunt, (7) can be deployed through an aortic graft, e.g., Dacron graft, and (8) can be used in adult and pediatric patients.

### BRIEF DESCRIPTION OF THE DRAWINGS

FIG. 1A depicts an embodiment of the filtration device having a filter retained by a sheath.

FIG. 1B depicts expansion of the filter of FIG. 1A.

FIG. 1C depicts an embodiment of the filtration device wherein the filter is collapsed by a distal capture sheath.

FIG. 1D depicts collapse of the filter of FIG. 1C.

FIG. 2A depicts deployment of the filter of FIG. 1B in the left iliac artery.

FIG. 2B depicts deployment of the filter of FIG. 1B in the right and left iliac arteries.

FIG. 3A depicts another embodiment of the filtration device having a filter mounted on a wire.

FIG. 3B depicts expansion of the filter of FIG. 3A.

FIG. 3C depicts cross sectional view of the device of FIG. 3B through section line C-C.

FIG. 4A depicts the filter of FIG. 3B deployed in an antegrade direction in the right and left iliac arteries.

FIG. 4B depicts deployment of the filter of FIG. 3B in the left iliac artery.

FIG. 4C depicts deployment of the filter of FIG. 3B in the lower abdominal aorta.

FIG. 4D depicts deployment of a filtration device in the abdominal aorta during a minimally invasive procedure.

FIG. 5A depicts another embodiment of the filtration device having first and second filters.

FIG. 5B depicts the device of FIG. 5A inserted in the left iliac artery having a collapsed first filter and an expanded second filter.

FIG. 5C depicts the device of FIG. 5A inserted in the left iliac artery having an expanded first filter and a collapsed second filter.

FIG. 6A depicts the point of attachment of a filter mesh at the middle of the expansion frame.

FIG. 6B depicts the point of attachment of a filter mesh at the proximal region of the expansion frame.

FIG. 7 depicts a filter having a windsock design that allows aspiration of emboli into a sheath.

FIG. 8A depicts the filtration device of FIG. 3B inserted in an arterial shunt.

FIG. 8B depicts the filtration device of FIG. 3B inserted through the shunt into the lower abdominal aorta.

FIG. 8C depicts filtration devices deployed in the iliac arteries through the shunt of FIG. 8A.

FIG. 9 depicts the filtration device of FIG. 3B inserted into the aorta through a prosthetic graft.

FIG. 9A depicts the filtration device of FIG. 3B inserted into the prosthetic graft of FIG. 9.

FIG. 9B depicts filtration devices deployed in the iliac arteries through the prosthetic graft of FIG. 9.

FIG. 10 depicts another embodiment of the device having a sheath capable of being dilated into contact with the wall of the left iliac artery.

FIG. 11A depicts another embodiment of the filtration device having a distal capture sheath for collapsing the filter.

FIG. 11B depicts expansion of the filter of FIG. 11A.

FIG. 11C depicts filtration and concurrent aspiration using the filter of FIG. 11B.

FIG. 12A depicts another embodiment of the filtration device having a filter mounted on a distal end of an elongate member insertable within a sheath.

FIG. 12B depicts partial closure of the filter of FIG. 12A by withdrawing the elongate member proximally.

FIG. 12C depicts further closure of the filter of FIG. 12A by rotating the elongate member clockwise relative to the sheath.

FIG. 12D depicts the filter of FIG. 12A collapsed into the sheath.

FIG. 13A depicts the filtration device of FIG. 3B inserted in an aortoiliac bypass graft.

FIG. 13B depicts the filtration device of FIG. 3B inserted in the left external iliac artery through an aortoiliac bypass graft.

FIG. 13C depicts the filtration device of FIG. 3B inserted in an iliofemoral bypass graft.

FIG. 13D depicts the filtration device of FIG. 3B inserted in the left femoral artery through an iliofemoral bypass graft.

FIG. 13E depicts the filtration device of FIG. 3B inserted in a right aortorenal bypass graft and in the left distal renal artery through a left aortorenal bypass graft.

FIG. 13F depicts the filtration device of FIG. 3B inserted in the right common iliac artery through an ilioiliac bypass graft.

### DETAILED DESCRIPTION

Although the filtration devices disclosed herein are most suitable for insertion in the aorta, iliac and femoral arteries, it should be understood that the devices and methods can be used in any vascular procedures where distal embolization is likely to occur. The devices and methods will find use for example in the ascending aorta, the descending aorta, aortic arch, common carotid artery, external and internal carotid arteries, brachiocephalic trunk, middle cerebral artery, anterior cerebral artery, posterior cerebral artery, vertebral artery, basilar artery, subclavian artery, brachial artery, axillary artery, iliac artery, renal artery, femoral artery, popliteal artery, celiac artery, superior mesenteric artery, inferior mesenteric artery, anterior tibial artery, and posterior tibial artery.

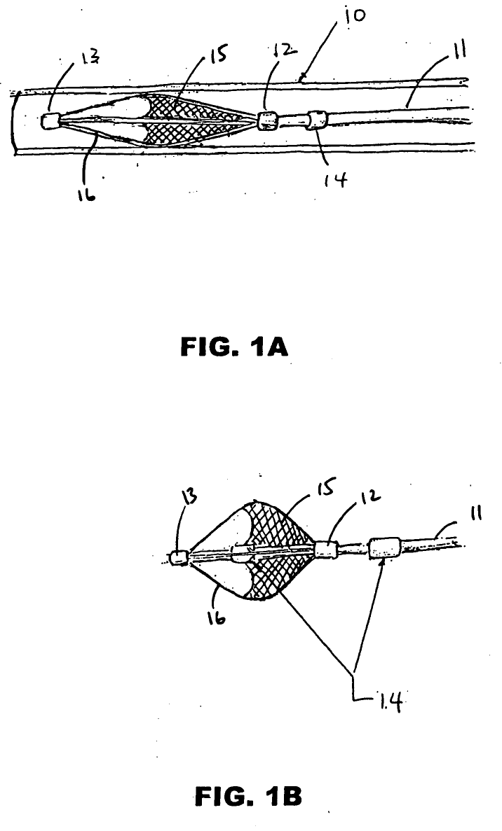

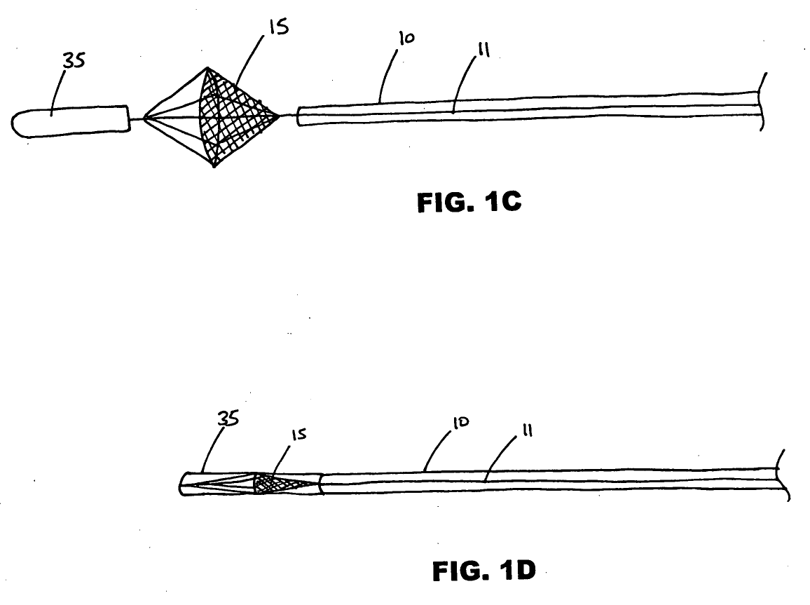

FIG. 1A depicts a device according to a first embodiment. The filtration device comprises elongate member 11 having filter 15 carried at a distal end. Struts 16 are bonded at a proximal end to proximal connector 12, and at a distal end to distal connector 13. Proximal connector 12 and distal connector 13 slide over elongate member 11 and are retained by proximal and distal stops 14. Sheath 10 covers and retains filter 15 and struts 16 before deployment. In certain embodiments, struts 16 are radially biased to be expanded when not contained within sheath 10. Struts 16 may be constructed of any suitable material, e.g., nitinol or stainless steel. FIG. 1B shows the expanded filter of FIG. 1A having sheath 10 removed. When filter 15 is pulled back into sheath 10, proximal connector 12 bears against distal stop 14. In other embodiments, distal capture sheath 35 is provided as shown in FIG. 1C. Distal capture sheath 35 assists with closing filter 15 as shown in FIG. 1D.

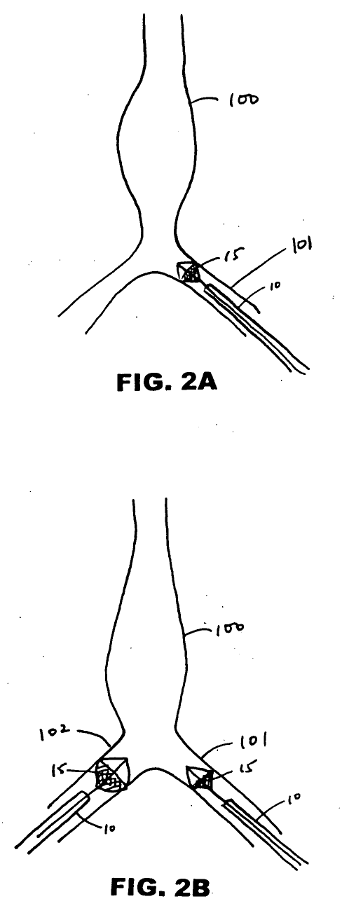

In use, the filter of FIG. 1A is inserted in a retrograde direction into an iliac artery as depicted in FIG. 2A. Sheath 10 is withdrawn, filter 15 is released, and filter 15 expands to cover the lumen of left iliac artery 101. The filter is thus expanded downstream of aortic aneurysm 100. FIG. 2B shows first and second filters 15, one disposed in left iliac artery 101 and the other deployed in right iliac artery 102. It will be understood that, during aortic aneurysm repair, it is desirable to protect both iliac arteries as shown in FIG. 2B or both femoral arteries.

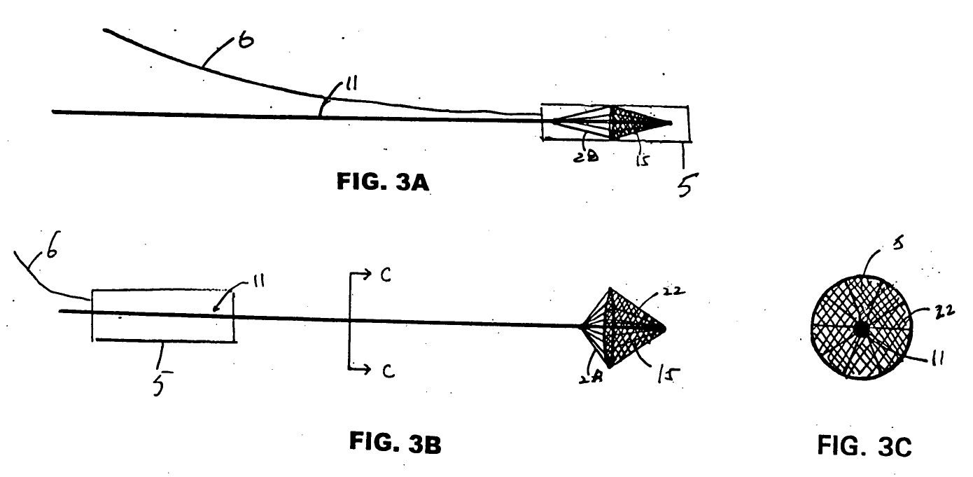

FIG. 3A depicts another filtration device adapted for use during aortic aneurysm repair. Elongate member 11 comprises a wire having struts 28 and filter 15 mounted at a distal end. Rapid exchange capture sheath 5 covers filter 15 and is attached to elongate member 6. In use, elongate member 6 is withdrawn to remove capture sheath 5 from filter 15, allowing the filter to expand as shown in FIG. 3B. FIG. 3C shows a cross-sectional view of the device of FIG. 3B taken through section line C-C.

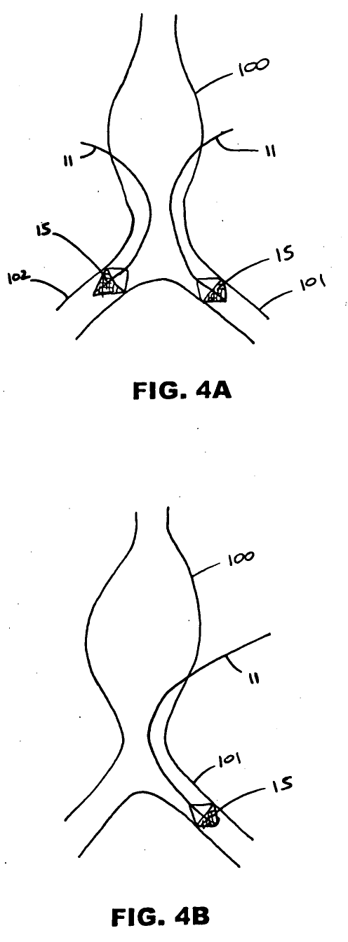

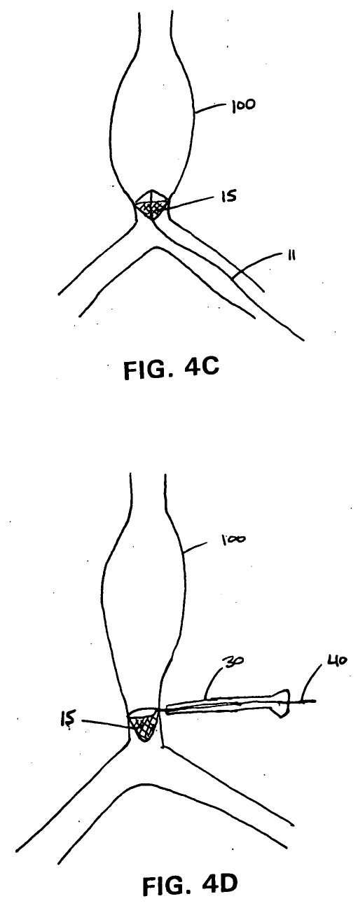

In use, one or more filters are inserted through the aneurysm and into the iliac arteries in an antegrade direction as shown in FIGS. 4A and 4B. FIG. 4A shows a first filter 15 mounted on a first wire 11 deployed within left iliac artery 101. FIG. 4A also shows second filter 15 mounted on second wire 11 expanded within right iliac artery 102. FIG. 4B shows only a single filter 15 deployed within left iliac artery 101. FIG. 4C shows filter 15 mounted on elongate member 11 inserted retrograde into the lower abdominal aorta downstream aortic aneurysm 100.

FIG. 4D depicts the deployment of a separately insertable filter 15 mounted at the distal end of elongate member 40 through introducer 30 into the lower abdominal aorta. In use, introducer 30 is inserted through the abdominal wall into the aorta. Filter 15 and elongate member 40 are inserted through introducer 30 until the filter enters the lumen of the lower abdominal aorta and expands. The aortic aneurysm is then repaired in accordance with methods described herein.

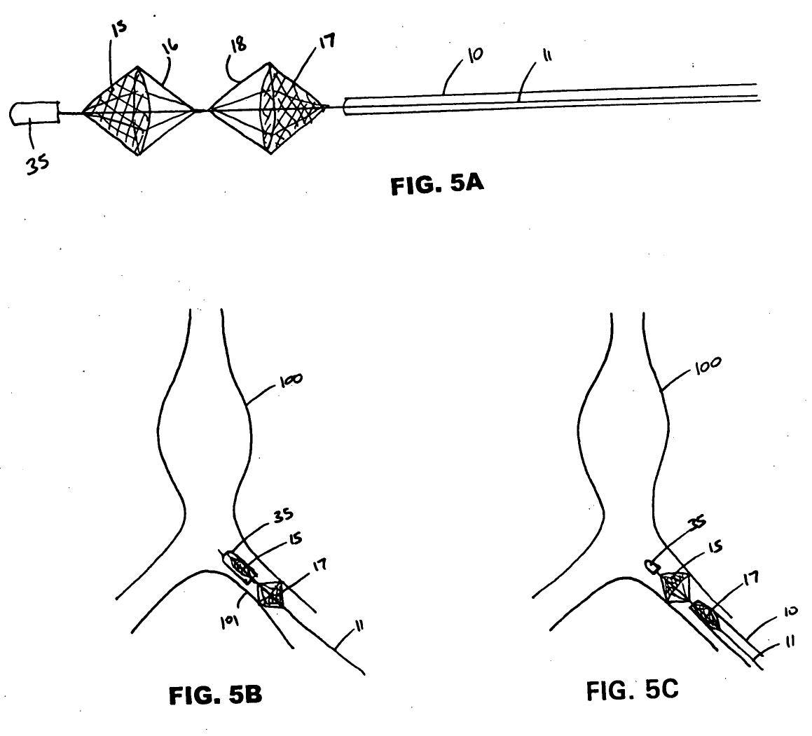

FIG. 5A depicts another medical device for distal protection during open surgical repair of an aortic aneurysm. The device of FIG. 5A is especially adapted for application in surgeries where blood flow within the lower abdominal aorta, iliac arteries, or femoral arteries is expected to reverse during a portion of the procedure. Thus, elongate member 11 carries first filter 15 fixed to struts 16 and second filter 17 fixed to struts 18. Both filters are carried at a distal end of wire 11. Proximal sheath 10 advances to cover filter 17 while distal capture sheath 35 is withdrawn to cover filter 15. Thus, each filter is equipped with a mechanism for expanding and contracting independently of the other filter.

In use, elongate wire 11 is located within left iliac artery 101 downstream aortic aneurysm 100 as shown in FIG. 5B. Distal capture sheath 35 covers and restrains filter 15. The proximal sheath is removed allowing filter 17 to expand and filter blood flowing from the aorta into the iliac arteries. If blood flow during the procedure. reverses within left iliac artery 101, sheath 10 is advanced to collapse filter 17, and distal capture sheath 35 is advanced to release filter 15 as shown in FIG. 5C.

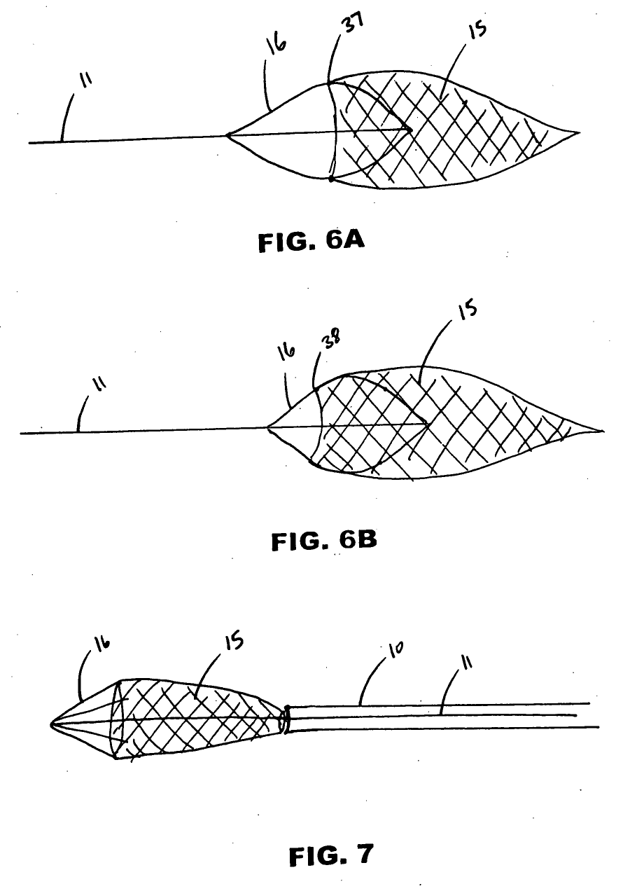

FIGS. 6A and 6B show mesh 15 having different points of attachment to struts 16 carried at the distal end of elongate member 11. In FIG. 6A, mesh 15 is attached substantially at midpoint 37 of struts 16. In FIG. 6B, mesh 15 is attached to struts 16 at a position 38 proximal of the midpoint. This allows a longer filter 15 to capture a large. embolic load.

FIG. 7 shows a windsock filter mechanism having aspiration capabilities. Sheath 10 carries struts 16 and filter 15 at a distal end. Filter 15 has an open distal end for entry of blood and emboli. The proximal end of filter 15 opens into a lumen of sheath 10. Emboli captured on filter 15 are aspirated into sheath 10 such that the filter does not carry the embolic load but instead flushes emboli into sheath 10. The emboli are passed through sheath 10 and are removed from the vessel.

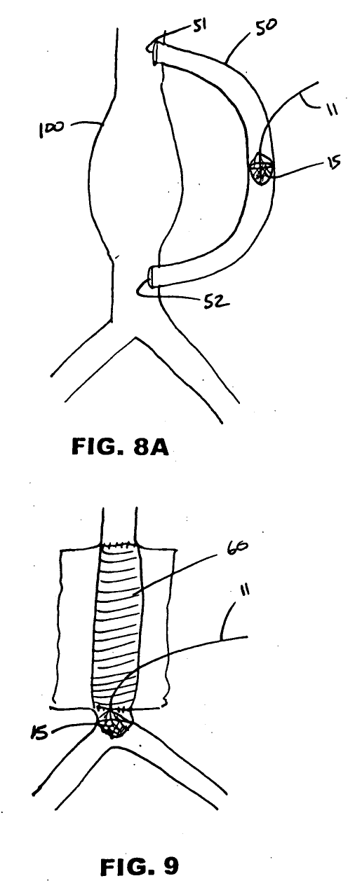

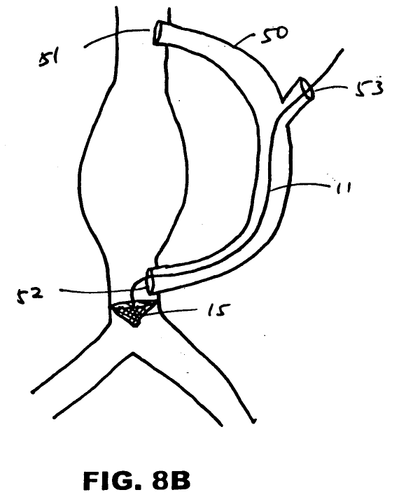

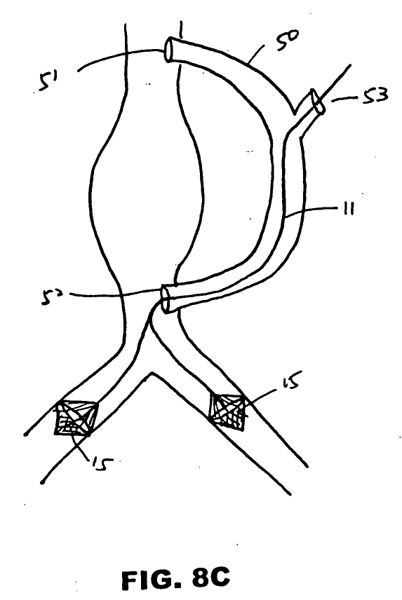

During aortic surgeries, a bypass graft is sometimes inserted upstream and downstream the aneurysm to redirect blood flow as depicted in FIG. 8A. Blood enters through proximal end 51 from the aorta and exits distal end 52 of graft 50 to perfuse downstream organs, e.g., the kidneys. Filter 15 may be inserted through a stick incision on the graft to capture emboli flowing through blood within the graft. Alternatively, as shown in FIG. 8B, a shunt is provided having a branching port 53 at an intermediate position along the shunt. Elongate member 11 passes through port 53, enters tubular member 50, passes through distal port 52, and is located and expanded within the lower abdominal aorta to capture and prevent embolic debris from traveling to the lower extremities. Another alternative method to prevent distal embolization is illustrated in FIG. 8C where filters 15 are inserted through branching port 53 of the shunt and deployed in the right and left iliac arteries. Filters 15 may be carried on elongate member 11 and inserted simultaneously through the shunt, or they may be mounted on separate elongate members and inserted independently through the shunt to deploy in the iliac arteries.

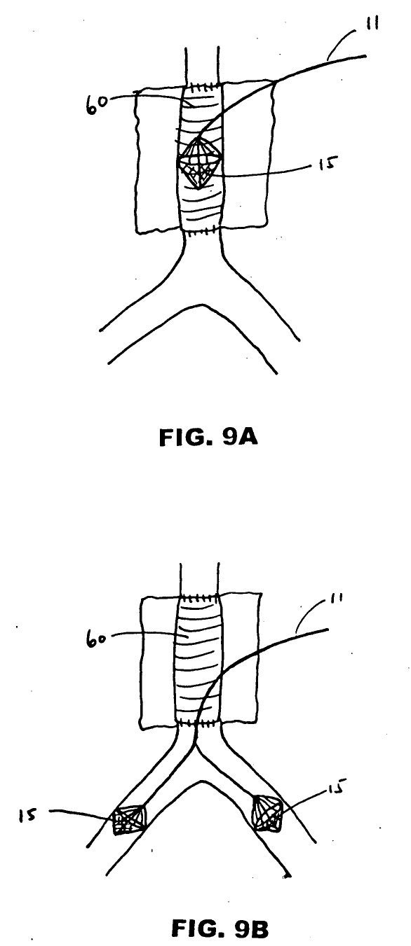

During a typical abdominal aortic aneurysm repair, a midline incision is made in the mid abdomen. The abdominal aorta is exposed and mobilized. Arterial clamps are placed on the aorta above the region of interest and on the iliac arteries below. The inferior mesentery artery, which is usually obliterated at its origin from the aorta, is ligated and divided. The aneurysm is then incised. The anterior portion of the aneurysm is removed and excess aneurysmal tissue is trimmed away. A preclotted prosthetic graft, e.g., woven Dacron, is inserted and sutured end to end into the aorta. The remaining aneurysmal wall is then sutured around the Dacron graft, the posterior peritoneum is closed, and arterial clamps are released to re-establish blood flow. In using the filtration device described in FIG. 3B to prevent distal embolization during the AAA repair, an incision is made on the prosthetic graft prior to closure of the aneurysmal wall. As depicted in FIG. 9, filter 15 is then inserted in the aorta through Dacron graft 60 prior to the release of arterial clamps. Embolic debris generated during the procedure is captured and prevented from traveling into the lower extremities when blood flow is re-established. After filter 15 is collapsed and removed from the aorta, the incision in Dacron graft 60 is sutured, the aneurismal wall is sutured around the Dacron graft, and the posterior peritoneum is closed. Alternatively, filter 15 is inserted and deployed in Dacron graft 60 to capture embolic debris as shown in FIG. 9A. Another alternative method to prevent distal embolization is illustrated in FIG. 9B where first and second filters 15, carried on one or more elongate member 11, are inserted through Dacron graft 60 and deployed in the right and left iliac arteries.

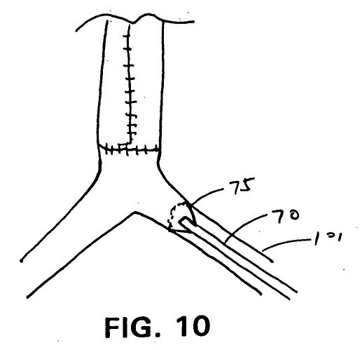

FIG. 10 depicts another embodiment of the device capable of preventing distal embolization without using a blood filter. The device comprises elongate tubular member 70 having a lumen that communicates with expandable membrane 75 at its distal end. Membrane 75 is collapsed to facilitate insertion into the vessel and is expanded into contact with the wall of left iliac artery 101 prior to release of the arterial clamps. A second expandable membrane can be deployed in the right iliac artery to protect both sides. Arterial clamps can be released for a few seconds to release embolic debris into expanded membrane 75. Embolic debris, blood, and/or fluid can be aspirated through membrane 75 and the lumen of elongate tubular member 70. Fluid, such as saline or lactated Ringer's solution, can be infused through the lumen of elongate tubular member 70 to irrigate the lower abdominal aorta and iliac arteries. With repeated irrigation and aspiration, embolic debris generated during the aortic procedure can be removed and prevented from traveling downstream to the lower extremities. After re-establishment of blood flow, membrane 75 is collapsed and removed from the iliac artery.

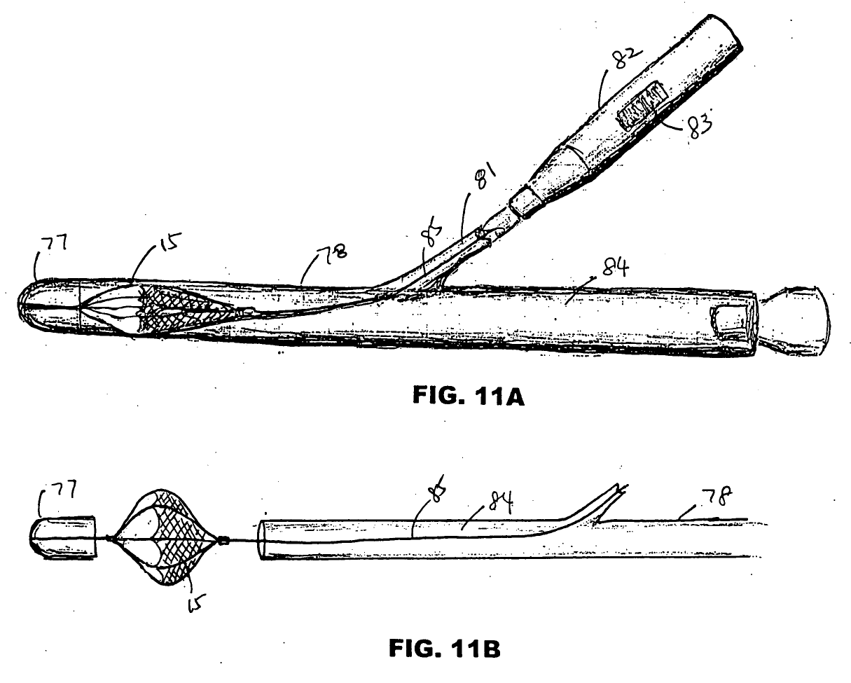

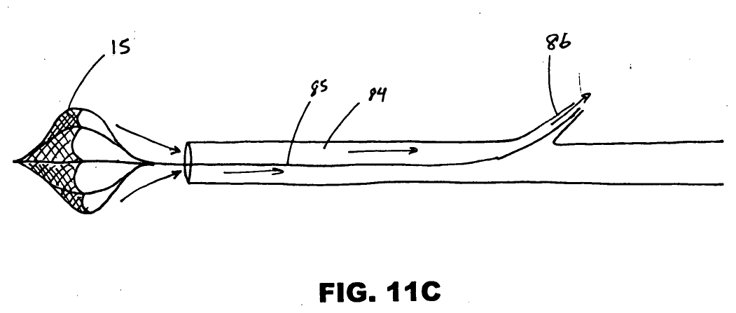

FIGS. 11A and 11B depict another embodiment of the filtration device having distal capture sheath 77. The device includes elongate tubular member 78 having first lumen 84 which is adapted for aspiration of fluid, blood, and/or embolic debris. First lumen 84 communicates with second lumen 81 that is adapted for insertion of filter 15. Filter 15 is mounted on a distal region of wire 85 that is attached to handle 82. The filter is opened and closed by operating knob 83 on handle 82. In FIG. 11A, filter 15 is collapsed by moving knob 83 proximally, thereby retracting distal capture sheath 77 and moving filter 15 into lumen 84. In FIG. 11B, filter 15 is expanded by moving knob 83 distally, thereby advancing capture sheath 77 and filter 15 distally. FIG. 11C depicts filtration with concurrent aspiration to reduce debris buildup. This function enables filter 15 to take on a larger load of emboli. Suction is applied to aspiration port 86. Port 86 may alternatively be used for drug delivery, for example to administer ReoPro, urokinase, or heparin.

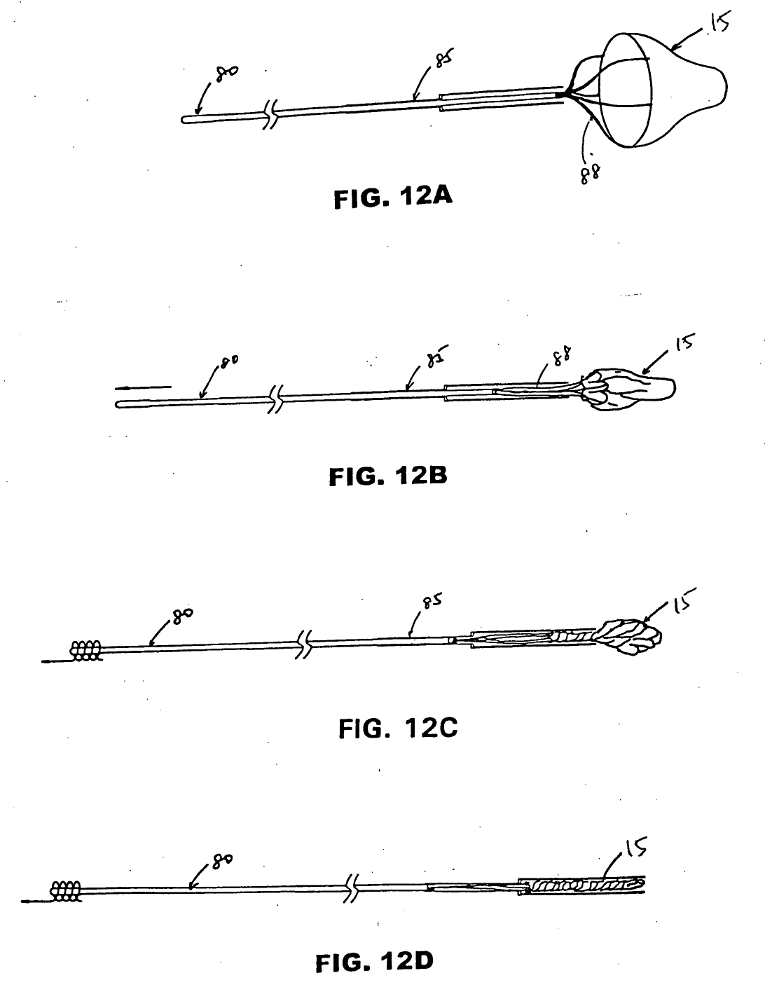

FIGS. 12A-12D depict another embodiment of the filtration device having filter 15 mounted on a distal end of elongate member 80 insertable through sheath 85. To collapse filter 15, elongate member 80 is withdrawn proximally relative to sheath 85, thereby retracting struts 88 into the sheath as shown in FIG. 12B. Elongate member 80 is then rotated clockwise relative to sheath 85 to collapse filter 15 as shown in FIG. 12C. As clockwise rotation continues, filter 15 and struts 88 are completely contained within sheath 80 as shown in FIG. 12D.

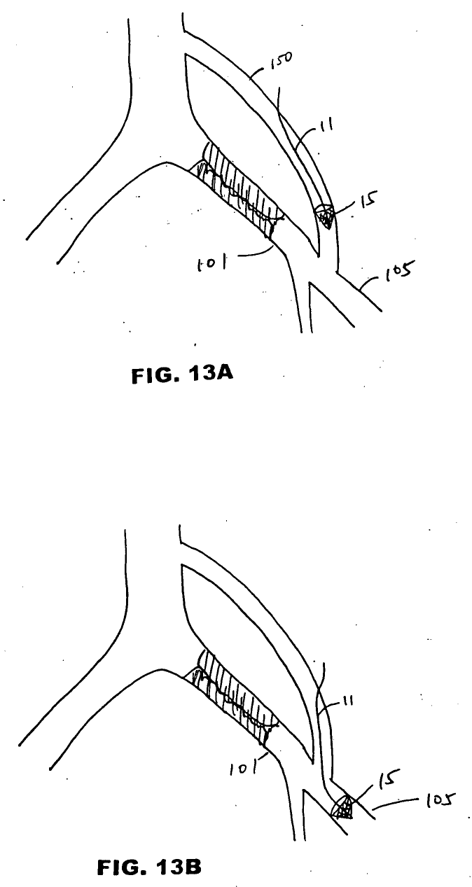

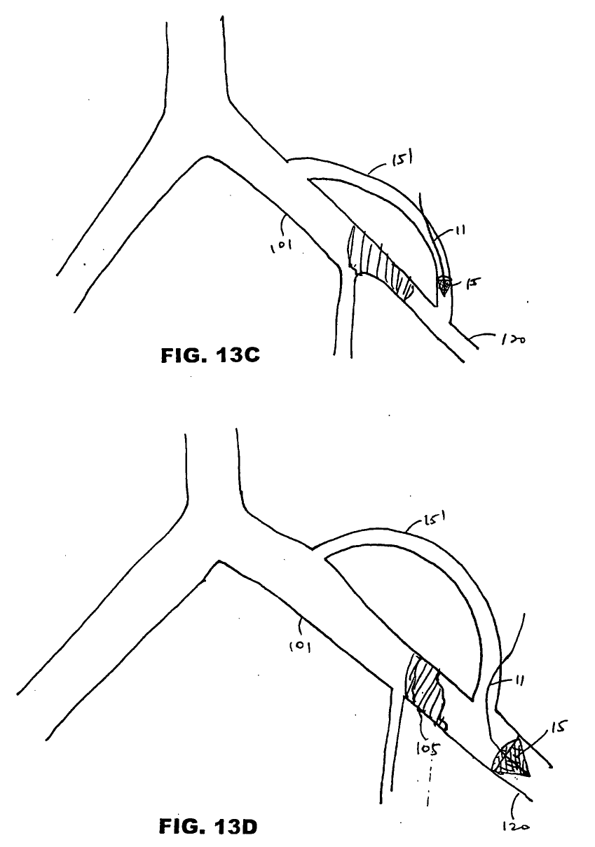

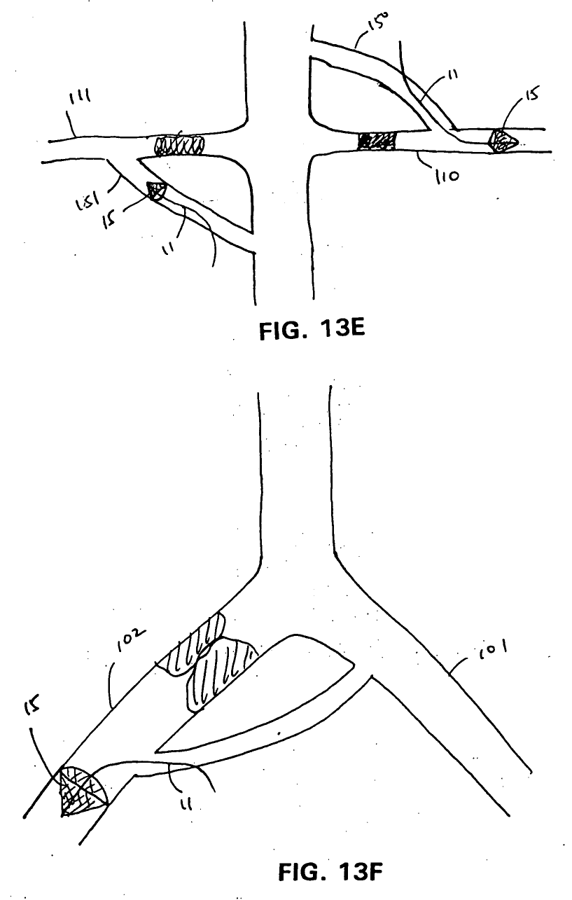

The filtration devices disclosed herein are also useful in treatment of occlusive vascular diseases involving the renal arteries and peripheral arteries. The surgical procedures useful in treating these vascular occlusions usually include thromboendarterectomy, or bypass graft using woven prosthetic tube or autogenous vein (e.g., saphenous vein) anastomosed end-to-side to the vessel above and below the obstruction. While Dacron is often the preferred material for an arterial prosthesis to bypass disease in the aortoiliac area, PTFE (Gortex®) is the synthetic of choice for bypassing the iliac, femoral, popliteal, or tibial obstruction. For example, Dacron graft 150, attached proximally in the aorta and distally in left external iliac artery 105 is used to bypass an obstruction in left common iliac artery 101 as shown in FIG. 13A. Filter 15 carried by elongate member 11 is inserted in aortoiliac graft 150 to capture embolic debris generated during the vascular procedure. Alternatively, filter 15 is inserted through graft 150 and deployed in left external iliac artery 105 to prevent distal embolization as shown in FIG. 13B.

In FIG. 13C, iliofemoral graft 151 is placed between left common iliac artery 101 and left femoral artery 120 to bypass an obstruction in the left external iliac artery. Saphenous vein or Gortex® is commonly used for the bypass graft. Filter 15 is inserted in iliofemoral graft 151 to capture embolic debris generated during the vascular procedure. Alternatively, filter 15 is inserted through graft 151 and deployed in left femoral artery 120 to prevent distal embolization as shown in FIG. 13D.

In treating renal artery stenosis, a graft may be placed between the aorta and the renal artery distal to the stenotic lesion. In FIG. 13E, graft 150 is placed between the aorta proximal the takeoff of the renal artery and left renal artery 110. Graft 151 is placed between the aorta distal the takeoff of the renal artery and right renal artery 111. Filter 15 may be inserted in the bypass graft (as shown in right aortorenal graft 151) or in the renal artery through the bypass graft (as shown through left aortorenal graft 150) to prevent distal embolization of vascular debris to the kidney.

FIG. 13F depicts a graft placed from left iliac artery 101 to right iliac artery 102 to bypass an occluding lesion in the proximal right iliac artery. Filter 15 is inserted through the graft and deployed downstream right iliac artery 102 distal the occlusion to prevent distal embolization. It will be understood that the filter devices disclosed herein can be used to prevent distal embolization during treatment of a variety of peripheral vascular diseases of the extremities including thrombectomy, endarterectomy, embolectomy, and bypass graft surgeries (e.g., aorto-bifemoral bypass, axillofemoral bypass, femoral popliteal bypass, and femorotibial bypass)

The length of the elongate member will generally be between 10 and 100 centimeters for aortic use, preferably approximately between 20 and 50 centimeters. The outer diameter of the sheath will generally be between 0.2 and 1.2 centimeters, preferably approximately between 0.4 and 0.8 centimeters. The filter will be capable of expanding to an outer diameter of at least 0.5 centimeters, more preferably at least 1.0 centimeter, more preferably at least 2.0 centimeters, more preferably at least 3.0 centimeters, more preferably at least 4.0 centimeters, more preferably at least 5.0 centimeters. The filter will be capable of contracting to an outer diameter of between 0.05 and 2.0 millimeters, preferably approximately between 0.8 and 1.2 millimeters. These ranges cover suitable diameters for both pediatric and adult use. The foregoing ranges are set forth solely for the purpose of illustrating typical device dimensions. The actual dimensions of a device constructed according to the principles of the present invention may obviously vary outside of the listed ranges without departing from those basic principles.

Although the foregoing invention has, for the purposes of clarity and understanding, been described in some detail by way of illustration and example, it will be obvious that certain changes and modifications may be practiced which will still fall within the scope of the appended claims. Moreover, it will be understood that each and every feature described for any given embodiment or in any reference incorporated herein, can be combined with any of the other embodiments described herein.

1. A method for open surgical repair of an aortic aneurysm, comprising the steps of:
inserting a filter into a vessel downstream of an aortic aneurysm;
expanding the filter;
repairing the aortic aneurysm; and
collapsing and removing the filter, wherein the filter captures emboli while the aneurysm is being repaired.

2. The method of claim 1, wherein the filter is inserted in a retrograde direction.

3. The method of claim 1, wherein the filter is inserted in an antegrade direction.

4. The method of claim 1, wherein the aortic aneurysm is an abdominal aortic aneurysm.

5. The method of claim 1, wherein the filter is expanded in the aorta downstream of the aortic aneurysm.

6. The method of claim 1, wherein the filter is expanded in a first iliac artery.

7. The method of claim 6, further comprising the step of expanding a second filter in a second iliac artery.

8. The method of claim 1, wherein the filter is expanded in a first femoral artery.

9. The method of claim 8, further comprising the step of expanding a second filter in a second femoral artery.

10. The method of claim 1, wherein the step of repairing the aortic aneurysm further comprises the steps of:
clamping the aorta above the aneurysm;
clamping the iliac arteries;
incising the aortic aneurysm; and
inserting and securing a prosthetic graft into the aorta.

11-20. (canceled)

21. A method for open surgical repair of an aortic aneurysm, comprising the steps of:
providing a shunt comprising a tubular member having a proximal end, a distal end, and a lumen therebetween, the tubular member branching and communicating with a side port located between the proximal end and the distal end;
inserting the proximal end of the shunt into the aorta upstream of the aneurysm;
inserting the distal end of the shunt into the aorta downstream of the aneurysm;
inserting a filter through the side port of the shunt and advancing the filter into the aorta downstream of the shunt;
expanding the filter; and
repairing the aortic aneurysm.

22-31. (canceled)

32. A method for open surgical repair of an aortic aneurysm, comprising the steps of:
providing a graft comprising a tubular member having a proximal end, a distal end, and a lumen therebetween;
attaching the proximal end of the graft into the aorta at the superior portion of the aneurysm;
attaching the distal end of the graft into the aorta at the inferior portion of the aneurysm;
deploying a filter downstream at least a portion of the aortic aneurysm; and
repairing or bypassing the aortic aneurysm.

33-43. (canceled)

44. A method for open surgical repair of an aortic aneurysm, comprising the steps of:
providing an elongate tubular member having a proximal end, a distal end, and a lumen therebetween, the distal end having an expandable membrane;
inserting the expandable membrane into a vessel downstream of an aortic aneurysm;
expanding the membrane; repairing the aortic aneurysm;
aspirating emboli through the lumen of the elongate tubular membrane; and
collapsing and removing the expandable membrane, wherein the membrane captures emboli while the aneurysm is being repaired and the emboli are removed through the lumen of the elongate tubular member.

45-47. (canceled)
